(19) United States
(12) Patent Application Publication
(10) Pub. No.: US 2007/0225755 A1
(43) Pub. Date: Sep. 27, 2007

(54) Closure device

(75) Inventors: Fredrik Preinitz, Uppsala (SE); Per Egnelov, Phuket (TH); David Fallman, Uppsala (SE)

Correspondence Address: FOLEY AND LARDNER LLP;SUITE 500, 3000 K STREET NW, WASHINGTON, DC, 20007, US

(73) Assignee: RADI MEDICAL SYSTEMS AB

(21) Appl. No.: 11/386,081

(22) Filed: Mar. 22, 2006

(51) Int. Cl. A61B 17/08

(52) U.S. Cl. 606/213

(57) ABSTRACT

A medical closure device (20) is provided, comprising a tubular member (21) provided with first struts (22) that extend between a first end portion (24) and a central portion (25) and second struts (23) that extend between said central portion (25) and a second end portion (26), each strut being provided with a hinge section (27, 28), such that said closure device is movable between a first elongated introduction configuration and a second positioning configuration in which the first and/or second end portions have been moved towards each other such that the first and/or second struts have moved radially away from a longitudinal central axis of the closure device. The closure device has further a third configuration, in which a locking member (30) is positioned in the tubular member such that the first end portion abuts a first end rim (32) of the locking member and an enlarged second end rim (36) of the locking member prevents further compression of the tubular member, and the closure device has a fourth configuration in which the second end portion has been moved over the second end rim such that the closure device is held in an expanded and locked configuration.

The present invention relates generally to a medical device for closing an opening or defect in an organ within a living body, e.g. a septal defect in a heart or a percutaneous puncture in a vessel wall (such as walls in arteries, or other blood vessels), and in particular to an expandable and repositionable closure device, which can be remotely maneuvered from an initial positioning configuration to a final configuration in which the opening or defect is closed.

### BACKGROUND OF THE INVENTION

The closing of an opening in an organ of a patient is a medical procedure that frequently has to be practised by doctors or other trained medical personnel. The opening may be a hole created by the doctor for a specific and usually temporary purpose, or the opening can be a congenital or acquired defect. An example of the former would be a puncture hole created in a patient's femoral artery to obtain access to the coronary system, while an example of the latter is a septal defect in a patient's heart. For descriptive and illustrative purposes the present invention will be described with reference to such a septal defect, although such techniques can be applied to other fields of application.

As is well-known, the human heart is divided into four chambers: the left atrium, the right atrium, the left ventricle, and the right ventricle. The atria are separated from each other by the interatrial septum, and the ventricles are separated by the interventricular septum.

Either congenitally or by acquisition, abnormal openings or holes can form between the chambers of the heart, causing shunting of blood through the opening or hole. For example, with an atrial septal defect, blood is shunted from the left atrium to the right atrium, which produces an overload of the right side of the heart. In addition to left-to-right shunts such as occur in patent ductus arteriosus from the aorta to the pulmonary artery, the left side of the heart has to work harder because some of the blood will recirculate through the lungs instead of going to the rest of the body. The ill effects of such lesions usually cause added strain on the heart with ultimate failure if not corrected.

One way to cure a septal defect in the septum of a heart is to position and anchor a specially designed closure device at the septum such that both sides of the septal defect are spanned by the closure device to thereby close the defect. Examples of such septal defect closure devices are known from the U.S. Pat. Nos. 5,853,422; 6,024,756; 6,117,159 and 6,312,446 to Huebsch et al., which disclose a closure device comprising a cylindrical shaft of metal or polymeric material with concentric parallel cuts through the wall of the device to thereby create flattened support struts. The centers of the support struts are intended to move radially away from the longitudinal axis of the device in a hinge like fashion in response to movements of the proximal and distal ends of the device towards the centre thereof. The closure device is claimed to be reversibly operable between a delivery configuration and a defect closing configuration, in which the closure device can be locked.

A similar septal defect closure device is also disclosed in the international application WO 2005/006990 A2.

### SUMMARY OF THE INVENTION

In the patents cited above, no means are, however, provided to create a well-defined, user-perceivable transition between the closing configuration and the locked configuration. Within the medical field it is of utmost importance that closure devices work properly, and a general object of the present invention is therefore to improve a closure device of the aforementioned type in such a way that a safe and user-friendly medical device is obtained, whose movements and configurations can be sensed and controlled in a more reliable way in comparison with the previously known devices.

According to the present invention, a closure device comprises an elongated tubular member in which a first set of longitudinal slits or cuts has been made on a first side of a shorter uncut central portion and a second set of longitudinal slits or cuts has been made on the opposite side of the central portion. On each side of the central portion, the slits extend towards the ends of the tubular member to terminate a short distance before the respective end, such that uncut proximal and distal end portions are formed. The tubular member, which is made from a flexible and preferably resorbable material, has thereby been provided with proximal and distal sets of struts or ribs. The distal ends of the distal struts are flexibly connected to the distal end portion of the tubular member, while the proximal ends of the distal struts are flexibly connected to the central portion. Similarly, the proximal ends of the proximal struts are flexibly connected to the proximal end portion of the tubular member, while the distal ends of the proximal struts are flexibly connected to the central portion. The struts are further each provided with a weakened section, which can act as a hinge, such that each strut in effect is divided into two articulated arms.

When the closure device during use is compressed such that the distal and proximal end portions are forced towards each other, the weakened sections of the struts move radially out from the longitudinal central axis of the closure device, and the respective arms of the struts assume an essentially perpendicular angle to the central axis of the closure device. The closure device comprises further a central cylindrical locking member, which preferably is separate from the tubular member and which over its length comprises several portions with different diameters. In use, the cylindrical locking member is inserted into the tubular member such that the distal end portion of the tubular member abuts a distal end rim of the locking member, and the proximal end portion of the tubular member is then pushed over a proximal end rim of the locking member. In the compressed state, the central, proximal and distal portions of the tubular member fit snugly over respective portions of the central locking member, and the closure device is held in the compressed state by the enlarged distal and proximal rim portions of the locking member, which prevents the closure device from resuming its original elongated shape.

In accordance with the present invention, the closure device has thereby been provided with four (4) well-defined configurations: an introduction configuration, a positioning configuration, a closed configuration, and a locked configuration, which are all well-defined and, more importantly, are easily discernible by a doctor who is implanting the closure device in, for example, the septum of a patient's heart. The usefulness of having well-defined configurations will be apparent from the detailed description below taken in conjunction with the appended drawings.

### BRIEF DESCRIPTION OF THE DRAWINGS

FIG. 1 is a schematic illustration of a human heart having an atrial as well as a ventricular septal defect.

FIG. 2 is a schematic illustration of a human heart having a septal defect, which is to be closed by means of a medical procedure that, in a first step, involves the introduction of a septal defect closure device according to the present invention.

FIG. 3 illustrates an intermediate step in the medical procedure, in which a distal portion of the closure device of FIG. 2 is expanded in order to locate the septal defect from the distal side of the septal defect.

FIG. 4 illustrates another intermediate step in the medical procedure, in which a proximal portion of the closure device of FIG. 2 is expanded in order to locate the septal defect from the proximal side of the septal defect.

FIG. 5 illustrates the closure device of FIG. 2, which has been positioned in the septum to close the septal defect therein.

FIG. 6 shows a septal defect closure device according to the present invention in an introduction configuration before any longitudinal compression of the closure device.

FIG. 7 shows the closure device of FIG. 6 in an intermediate semi-compressed positioning configuration.

FIG. 8 shows the closure device of FIG. 6 in another intermediate semi-compressed positioning configuration.

FIG. 9 shows a locking member, which constitutes a separate part of a septal defect closure device.

FIG. 10 shows the closure device of FIG. 6 in a closed configuration.

FIG. 11 shows the closure device of FIG. 6 in another closed configuration, in which distal portions of a mechanical actuator are visible.

FIG. 12 illustrates the closure device of FIG. 6 in a final locked configuration.

### DETAILED DESCRIPTION OF PREFERRED EMBODIMENTS OF THE INVENTION

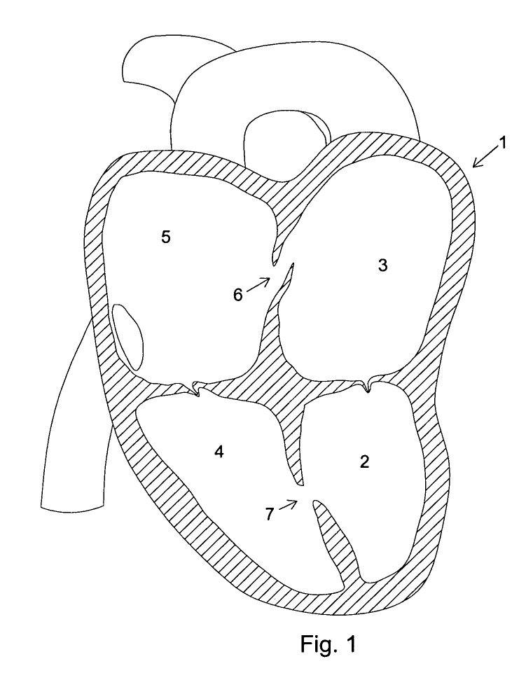

A schematic cross-sectional view of a human heart 1 is shown in FIG. 1. The heart 1, with its left ventricle 2, left atrium 3, right ventricle 4, and right atrium 5, suffers from an atrial septal defect 6 as well as a ventricular septal defect 7. Below a medical procedure will be discussed in which an atrial septal defect is closed. It should, however, be clear that a septal defect closure device according to the present invention equally well could be employed to close a ventricular septal defect like ventricular septal defect 7 of FIG. 1. It should further be noticed that the septal defects 6, 7 can be accessed from different vessels, e.g. from the superior or inferior vena cava, or from the aorta. This implies, in turn, that throughout the present description terms like “distal” and “proximal” should always be seen from the end of a delivering catheter, through which a septal defect closure device is delivered (and not from any particular chamber or vessel of a heart).

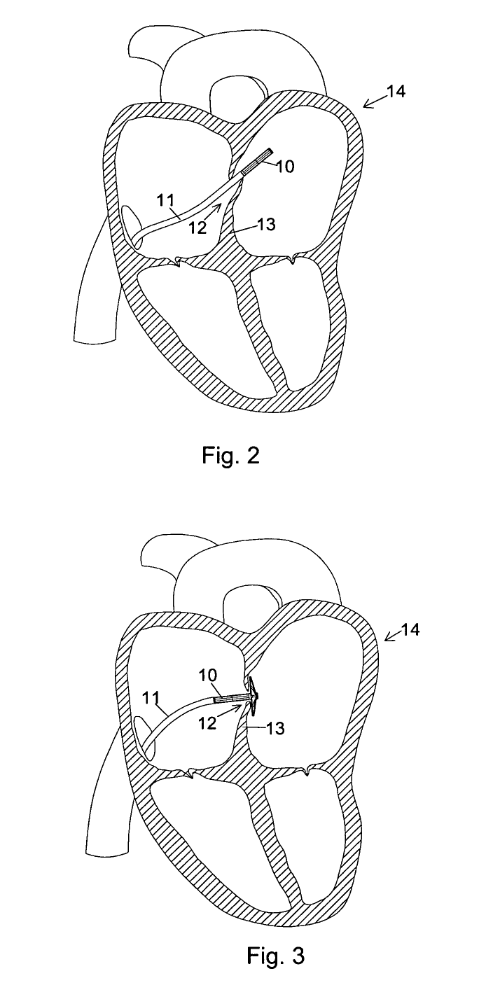

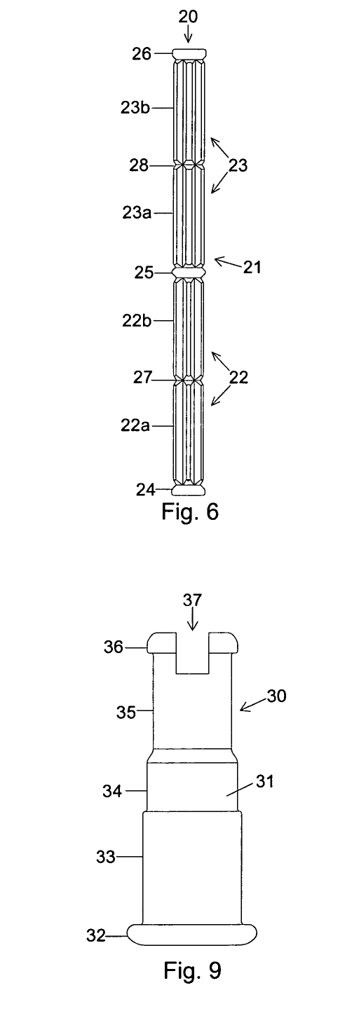

In conjunction with FIGS. 2 to 5, a medical procedure will be briefly described, in which a septal defect closure device according to one embodiment of the present invention is employed to close a septal defect in the septum of a heart; and thereafter different configurations and parts of the closure device itself will be described in detail in conjunction with FIGS. 6 to 12.

FIG. 2 illustrates a septal defect closure device 10 according to the present invention, which by means of a delivering catheter 11 has been introduced into an atrial septal defect 12 in the atrial septum 13 of a heart 14. The closure device 10 is of the same general construction that has been generally described above, and comprises an elongated tubular member in which distal and proximal sets of struts have been provided. The distal struts extend from a central portion of the closure device 10 to a distal end portion thereof, and the proximal struts extend from a proximal end portion of the closure device 10 to the central portion. As already discussed, each strut is provided with a thinner and thereby weaker section that can act as a hinge, and each strut is thereby effectively divided into two hinge-connected arms. In FIG. 2, the closure device 10 is shown in an initial introduction configuration, in which the arms of each strut are substantially aligned with each other. In this introduction configuration, the closure device 10 has therefore a generally elongated tubular shape, which facilitates the introduction of the closure device 10 into the artery and heart of a patient. The introduction configuration is defined as the configuration that the closure device assumes by itself, i.e. without any compression being induced by a mechanical actuator (not shown in the figure) connected to the closure device. In this introduction configuration, the closure device has therefore a generally tubular shape, although the closure device could be preformed such that the arms of each strut exhibit a small positive angle in relation to each other. Such a positive angle guarantees the proper radial expansion of the tubular member during longitudinal compression of the tubular member.

To ascertain correct positioning of the closure device 10 with respect to the septal defect 12, the distal set of struts can be moved radially outwards from the central axis of the closure device 10, such that a partly expanded configuration is obtained. The radial movements of the distal struts are effectuated by partially compressing the closure device 10 through the maneuvering of a mechanical actuator (not shown in FIGS. 2-5). In this semi-expanded locating or positioning configuration, the closure device 10 is retracted until the distal struts abut the distal side of the atrial septum 13 surrounding the septal defect 12. The septal defect 12 can thereby be located by a doctor, who in this phase of the medical procedure will feel a marked increase in resistance against further retraction. This intermediate step of the medical procedure is depicted in FIG. 3.

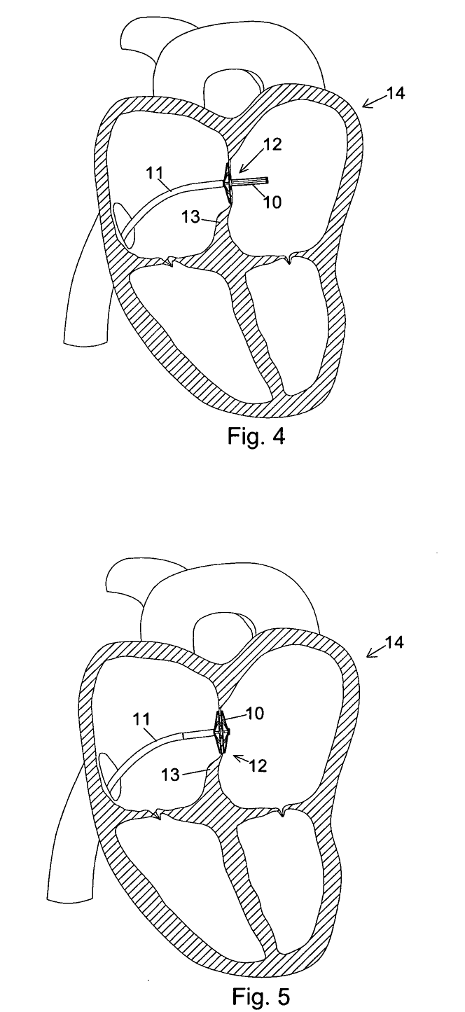

As an alternative, or complement, the proximal set of struts can be moved radially outwards from the central axis of the closure device 10, such that another partly expanded positioning configuration is obtained. As before, the radial movements of the proximal struts are accomplished by partially compressing the closure device 10 through the maneuvering of the mechanical actuator mentioned above. In this second semi-expanded locating or positioning configuration, the closure device 10 is advanced until the proximal struts abut the proximal side of the atrial septum 13 surrounding the septal defect 12. The septal defect 12 can thereby be located by a doctor who in this phase of the medical procedure will feel a marked increase in resistance against further advancement. This intermediate step of the medical procedure is depicted in FIG. 4. It may be mentioned that the closure device 10 can be reversibly moved between the elongated tubular introduction configuration of FIG. 2 and either of the intermediate positioning configurations shown in FIG. 3 and FIG. 4, respectively. The closure device 10 can also assume a further intermediate positioning or locating configuration in which the proximal struts as well as the distal struts have been moved radially outwards from the central axis of the closure device 10, as will be further discussed below.

When the atrial septum 13 and thereby the septal defect 12 have been correctly located, either by the step shown in FIG. 3 or by the step of FIG. 4, or by a combination of both steps, the closure device 10 is fully expanded such that the proximal struts as well as the distal struts are forced radially outwards by maneuvering of the mechanical actuator mentioned above. In this septal defect closing configuration, the closure device 10 spans both the distal side and the proximal side of the septal defect 12. As can be seen in FIG. 5, the closure device 10 sandwiches the atrial septum 13 to thereby close the septal defect 12 therein. It can be mentioned that the term “close” or similar terms used herein in conjunction with the description of the closing of a septal defect should not be taken too literally. Such terms are meant to encompass all stages from actually sealing or closing off a septal defect to merely restricting the flow of blood therethrough, the important thing being that the closure device permits and facilitates healing of the septal (or other type of) defect over time. To improve the sealing capability of a closure device of the present type, it is conceivable that the distal and/or proximal struts at least partly are covered by a thin membrane or formed integrally with a thin membrane, which preferably is made from a resorbable material. This feature may in particular be advantageous when the closure device is used to seal a puncture hole in a vessel wall.

A special feature of the closed configuration illustrated in FIG. 5 is that the closure device 10 still is repositionable. This means that by means of a mechanical actuator, the closure device 10 is reversibly movable between the configurations described above in conjunction with FIGS. 2-5, i.e. from the closed configuration of FIG. 5, to anyone of the intermediate positioning configurations of FIG. 3 or FIG. 4, and back to the original introduction configuration of FIG. 2. The closure device 10 can then be retracted out of the patient's body and be disposed, or can once again be positioned by repeating the steps illustrated above. The closed configuration of the closure device 10 is defined as the extreme end position of the different and gradually changing positioning configurations. In the closed configuration, essentially no further compression of the closure device 10 is possible while still having a reversibly movable closure device 10. The latter will be thoroughly discussed below.

In accordance with the present invention, a closure device encompasses a fourth configuration, in which the closure device is irreversibly locked. The transition from the closed configuration to this locked configuration is effectuated by the mechanical actuator mentioned above. A special feature of the present closure device is that a doctor will feel when the closed configuration has been reached, so that he or she can decide whether the mechanical actuator should be maneuvered such that the locked configuration is achieved. Having in mind that the closed configuration constitutes a situation from which the closure device can be removed, whereas the locked configuration implies a non-retrievable closure device, the importance of having a well-defined transition between these two states should be appreciated. Also this feature will be further discussed below.

An embodiment of a septal defect closure device 20 according to the present invention is illustrated in FIG. 6. FIG. 6 shows the closure device 20 in a first or introduction configuration in which the closure device 20 has the general shape of an elongated tubular member 21, through which a number of longitudinal, parallel cuts or slits have been made to thereby form a first or distal set of struts 22 and a second or proximal set of struts 23. The first strut set 22 extends between a first end portion 24 of the tubular member 21 and a central portion 25 thereof, while the second strut set 23 extends between the central portion 25 and a second end portion 26 of the tubular member 21. The first and second end portions 24, 26 as well as the central portion 25 are uncut and are shorter than the slit portions of the tubular member 21. Somewhere along the length of the first set of struts 22, the tubular member 21 has been provided with a circumferential weakened section 27 in that material has been removed (or weakened in another fashion) from this ring-shaped section of the tubular member 21. The weakened thinner section 27 of each strut 22 will thereby act as a hinge or articulation 27, which effectively divides each strut 22 into two articulated arms: a first or distal arm 22a and a second or proximal arm 22b. Similarly, the struts in the second set of struts 23 are each provided with hinge section 28, which in effect divides each strut 23 into two articulated arms: a first or distal arm 23a and a second or proximal arm 23b.

Here it should be emphasized that the term “tubular” is merely intended to indicate the general shape of an elongated, cylindrical member, which comprises a number of struts, the ends of which are connected to shorter ring-shaped members, and which in a first introduction configuration assumes a tubular shape. In other words, a tubular member, like tubular member 21, does not actually have to be cut or slit in order to create distal and proximal struts. On the contrary, a tubular member, having struts with weakened hinge-sections as well as ring-shaped central, distal and proximal end portions, can advantageously be directly produced in this form, e.g. by injection molding. Furthermore, the struts of a tubular member, like tubular member 21, do not have to be exactly aligned with each other. Instead, a tubular member can be preformed in such a way that the two arms of a strut exhibit an angled relation to each other, to thereby guarantee that the arms actually bend outwards during compression of the tubular member. Nevertheless, the definition of the introduction configuration is still the configuration or state wherein a closure device has not been subjected to any compression by means of a mechanical actuator. The introduction configuration may therefore also be regarded as the “natural” state of the closure device.

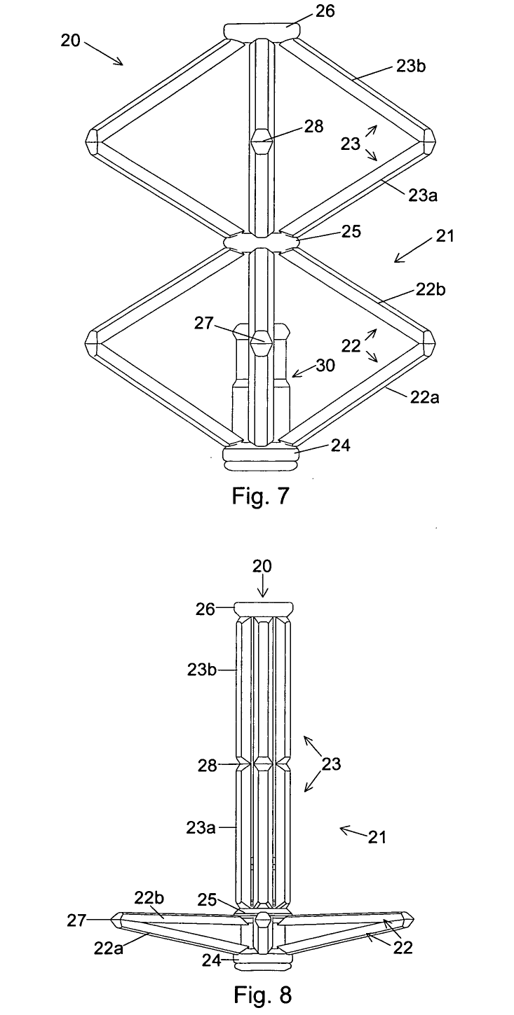

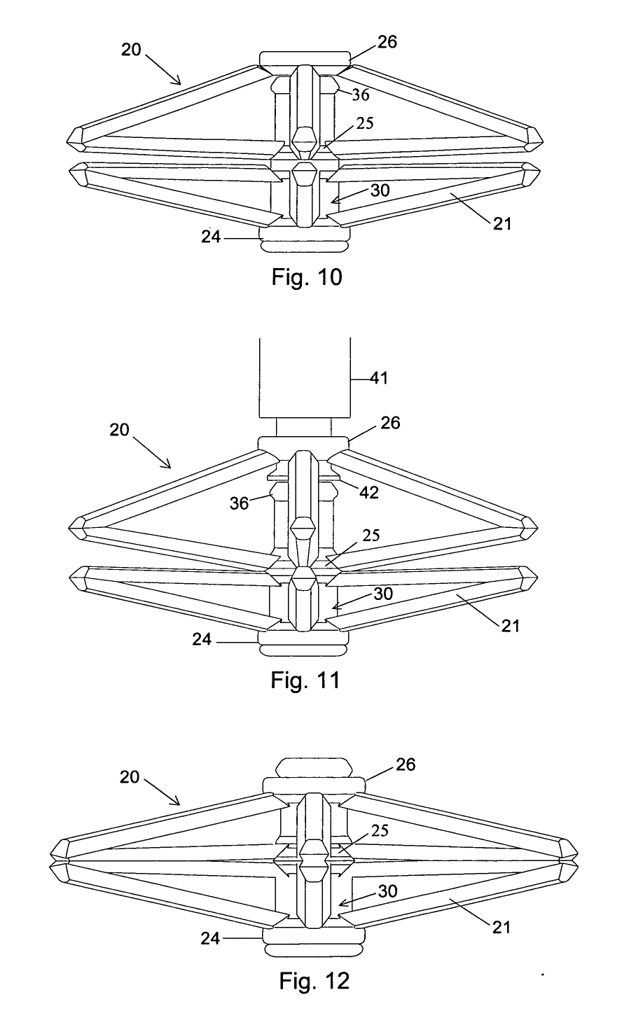

In FIG. 7, the closure device 20 of FIG. 6 is depicted in a semi-expanded positioning configuration, in which the distal and proximal end portions 24, 26 of the closure device 20 have been moved towards the central portion 25. The hinge sections 27, 28 of the first and second struts 22, 23 have thereby been forced to move outwards from the central axis of the closure device 20, and the articulated arms 22a, 22b and 23a, 23b have assumed an angled relation to the central axis of the closure device 20. Here it should be recognized that the configuration shown in FIG. 7 partly is for illustrative purposes; in practice either of the two end portions 24, 26 could be moved towards the central portion 25, to assume the locating configurations shown in FIG. 3 and FIG. 4, respectively. The semi-expanded configuration of FIG. 7 could, however, also be used to determine the proper position for the closure device 20, and can also be regarded as a positioning configuration prior to a closed configuration described below in conjunction with FIG. 10 or FIG. 11. The positioning configuration is consequently defined as all intermediate states between the introduction configuration defined above and the closed configuration, which will be described and defined below. Another example of a positioning configuration is illustrated in FIG. 8.

As can be seen in FIG. 7, the closure device 20 comprises further a locking member 30, which is separately illustrated in FIG. 9. The locking member 30, which according to the invention may constitute a separate part of closure device 20, or may be an integrated part thereof, comprises a hollow body 31, which along is length is provided with several portions with different outer diameters. More specifically, the body 31 of the locking member 30 comprises a distal end rim 32, a distal portion 33, an intermediate portion 34, a proximal portion 35, and a proximal end rim 36. The distance between the distal end rim 32 and the proximal end rim 36 is considerably smaller than the length of the tubular member 21. As the observant reader already may have appreciated, the respective outer diameters of the body 31 of the locking member 30 are related to the respective diameters of the tubular member 21 of the closure device 20. Thus, the diameter of the distal end rim 32 is larger than the inner diameter of the distal end portion 24 of the tubular member 20, while the inner diameter of the distal end portion 24 is larger than the other diameters of the body 31 of the locking member 30, such that the distal end portion 24 of the tubular member 21 can slide over the locking member 30 until the distal end portion 24 abuts the distal end rim 32. The outer diameter of the distal portion 33 of the locking member 30 is adapted to the inner diameter of distal end portion 24 of the tubular member 21, while the diameter of the intermediate portion 34 is adapted to the diameter of the central portion 25 of the tubular member 21. The inner diameter of the proximal end portion 26 of the tubular member 21 is adapted to the outer diameter of the proximal portion 35 of the locking member 30, and is slightly less than the diameter of the proximal end rim 36. During use, the proximal end portion 26 of the tubular member 21, which is made from a somewhat elastic material, must therefore be forced over the proximal end rim 36 and can then slide on the proximal portion 35. As can be seen in FIG. 9, the locking member 30 comprises preferably a recess 37, which provides the proximal end rim 36 with a certain resilience which facilitates the sliding of the proximal end portion 26 of the closure device 20 over the proximal end rim 36 of the locking member 30.

As indicated above, the closure device 20 can assume an infinite number of positioning configurations during a positioning operation in which a septal defect is located and the closure device 20 is positioned therein. According to the present invention, there is, however, a well-defined endpoint for the positioning operation. This endpoint, which is referred to as the closed configuration of the closure device 20, is illustrated in FIG. 10, where it can be seen that the central portion 25 of the tubular member 21 has been positioned over the intermediate portion of the locking member 30, while the proximal end portion 26 of the tubular member 21 abuts the proximal end rim 36 of the locking member 30. (For illustrative purposes only, there is a small gap between the proximal end portion 26 and the proximal end rim 36 in FIG. 10.) As has been mentioned above, the inner diameter of the proximal end portion 26 is slightly less than the diameter of the proximal end rim 36, which implies that further compression of the tubular member 21 is not possible—unless extra force is applied such that the proximal end portion 26 is forced over the proximal end rim 36. The closed configuration of FIG. 10 thereby constitutes a well-defined state.

The situation illustrated in FIG. 10 is, however, only one example of a closed configuration. In practice, the movements of the closure device are effectuated by the previously mentioned mechanical actuator, parts of an example of which are illustrated in FIG. 11 together with the tubular member 21 as well as the locking member 30. The mechanical actuator comprises a pusher tube 41 and an actuating member 42. By moving the actuating member 42 back and forth, a doctor can during a preceding positioning operation let the tubular member 21 assume different positioning configurations, to thereby locate a septal defect (or some other type of tissue opening, e.g. a percutaneous puncture in an artery wall) and position the closure device 20 in the opening of the defect. The movements of the tubular member 21 is actually accomplished in co-operation with a hold and release member (not shown in the figure), which releasably holds the locking member 30; and the compression of the tubular member is achieved by the relative motion between the hold and release member and the actuating member 42. In the situation illustrated in FIG. 11, the distal end of the actuating member 42 abuts the proximal end rim 36 of the locking member 30. (For illustrative purposes only, there is a small gap between the distal end of the actuating member 42 and the proximal end rim 36 in FIG. 11.) Thus, FIG. 11 illustrates a well-defined end position for the positioning operation, in which no further compression of the tubular member 21 is possible by maneuvering of the actuating member 42 in relation to the hold and release member without forcing proximal end portion 26 over proximal end rim 36. If, on the other hand, an actuating member were attached or engaged inside a proximal end portion of a tubular member, the situation would resemble the situation illustrated in FIG. 10, i.e. a well-defined end point of the positioning operation—in which no further compression of the tubular member is possible without forcing an end portion over an end rim—would be when a proximal end portion of the tubular member abuts a proximal end rim of a locking member. The closed configuration of a closure device according to the present invention is thereby defined as the extreme end position of the positioning configurations, wherein an end portion of a locking member prevents further compression of a tubular member. This definition also encompasses closure devices where a proximal end portion of a locking member prevents further compression of a tubular member, i.e. a closure device where a distal end portion of a tubular member is pulled over an enlarged distal end rim of a locking member rather than—as in the closure device described above—having a proximal end portion of a tubular member that is pushed over an enlarged proximal end rim of a locking member.

From FIG. 11 it may be realized that when the actuating member 42 abuts the proximal end rim 36 of the locking member 30, the closure device 20 can be transferred into the final locked state by movement of the pusher tube 41. To accomplish this, the pusher tube 41 (which can slide with respect to actuating member 42) is advanced, so that the proximal end portion 26 of the tubular member 21 is forced up and over the proximal end rim 36 of the locking member 30. This movement requires that the proximal end portion 26 and/or the proximal end rim 36 possesses a certain degree of resilience.

The final locked configuration of the closure device 20 is illustrated in FIG. 12, wherein the distal and proximal end portions 24, 26 of the tubular member 21 have been fully moved towards each other until the central portion 25 of the tubular member 21 is positioned over the intermediate portion of the locking member 30 and the proximal end portion 26 of the tubular member 21 has been moved over the proximal end rim 36 of the locking member 30. The closure device 20 is held in this compressed state due to the enlarged distal and proximal end rims 32, 36 of the locking member 30, which have diameters larger than the distal end portion 24 and the proximal end portion 26, respectively. The closure device 20 can then be released and left in this locked configuration by maneuvering of the hold and release member mentioned above. The locked configuration of a closure device is thereby defined as the configuration in which the closure device is fully expanded, and in which the closure device can be held without assistance of a mechanical actuator.

The septal defect closure device has been shown with proximal and distal struts having equal lengths. It is, however, possible to provide a closure device having proximal struts with one length and distal struts with a different length. It may, for example, be desirable to arrange a closure device in such a way that the left part of the closure device, i.e. the part that is implanted into the left atrium of a heart, is smaller than the right part of the closure device, to thereby reduce the amount of artificial material introduced into the left atrium, which in turn may reduce the formation of thrombogenic material therein. In this context, it should be recognized that it is not mandatory that a heart is accessed via the venous system, as is shown in FIGS. 2 to 5, but the heart could be accessed via the arterial side. This implies that if a doctor wishes to place a smaller part of a closure device at the left side of a heart than at the right side of the heart, then this smaller part (i.e. the shorter struts) will constitute the distal set of struts if the heart is accessed via the venous system, whereas the smaller part will constitute the proximal set of struts if the heart is accessed through the arterial system. It can therefore be appreciated that it can be advantageous to provide a closure device in the form of two separate tubular members (and a separate locking member) as this would provide a doctor with the possibility to tailor a septal defect closure device to the specific medical situation at hand, without the necessity of producing an excessive large number of closure devices with different dimensions.

It has already been mentioned that the length of the distal struts can differ from the length of the proximal struts; and it is also possible to have different lengths of the articulated arms within a strut set, such that, for example, the distal arms are longer than the proximal arms, or vice versa. The arms that actually contact a septum or a vessel wall can, for example, be shorter than the arms that do not contact the septum or the vessel wall, to thereby ensure a reliable closing of a septal defect in the septum or a puncture hole in the vessel wall.

It has already been mentioned that a locking member can constitute a separate part of a closure device, and a locking member can be made from a first material and a tubular member made from a second material. With different materials some specific advantages can be achieved. If, for example, the closure device is a resorbable closure device, then the resorption time of the material in the locking member can be different from the resorption time of the material in the tubular member, such that the locking force between the two members during the degradation of the closure device is reduced and ultimately lost in a controllable and predictable way. In this respect it may be advantageous if the material of the tubular member has a shorter resorption time than the material of the locking member. Further, whether or not the materials are resorbable materials, different requirements are put on the different pieces. For example, the material in the hinge portions of a tubular member must be flexible and have a high tenacity, whereas the locking member must have a rather high stiffness. Also in a resorbable closure device it can be necessary to have one material in a locking member and another material in a tubular member, because of the different dimensions involved. It can, for example, be necessary to have a material with a relatively long resorption time in the thin hinge portions of the tubular member in order to match the resorption time of the material in a thick-walled locking member.

Examples of resorbable materials for the tubular member and the locking member may include, but are not limited to, those materials made from aliphatic polyesters, polyether esters, and polycarbonates. More specifically, synthetic resorbable polymers such as homopolymers and copolymers made from any of the monomers lactide, glycolide, epsilon-caprolactone, trimethylene carbonate, and paradioxanone are advantageous because of their long clinical use.

The tubular member could preferably be made from a semi-crystalline material with a lower tensile modulus than the locking member. As previously stated, it could, e.g. because of the hinge portions, be an advantage to have a more flexible material in the tubular member. Such material is preferably made from a block copolymer characterized by having a soft middle part characterized by having a glass transition temperature below room temperature and a semi-crystalline part at each end of the soft middle part. The semi-crystalline part could be polymerized from any of the monomers glycolide, lactide, or paradioxanone. Since polyparadioxanone is a relatively soft and pliable material compared to polyglycolide and polylactide, the tubular member can be made from pure polyparadioxanone itself.

The locking member can be made from any of the above materials, but to secure the locking mechanism it is advantageous if the material is stiffer than the material used in the tubular member. The material should also preferably resorb at a somewhat slower pace than the tubular member. The locking member could also be made from amorphous or semi-crystalline material, and preferably from homopolymers or copolymers where the main monomer component is lactide, caprolactone, or paradioxanone.

A particular advantage of the groups of synthetic resorbable polymers mentioned above is that various mechanical properties can be accomplished by simply changing the monomer composition in the homopolymer or copolymer. Further, in contrast to natural biopolymers, these materials can be molded and machined into complex structures, and by varying the monomer composition large time spans can be achieved for their resorption times.

It may be appreciated that it can be advantageous to provide a radiopaque closure device which is visible in an X-ray machine. When the closure device is made from a synthetic resorbable polymer, a radiopaque closure device can conveniently be produced by mixing the polymer with suitable radiopaque agent. A suitable radiopaque agent is barium sulfate, which can be blended into the polymer or copolymer in an amount between 5% and 50%, and more preferably in an amount of 15% to 30%, to obtain the opacity needed in order to locate the closure device during an X-ray observation. Radiopaque materials can be used in a tubular member of the closure device, but is preferably used in the locking member, which marks the centre of the device. The radiopaque agent, e.g. barium sulfate, can—instead of being mixed with the polymer—be introduced into preformed holes in the closure device, which are then sealed by a synthetic resorbable material. As an alternative, preformed holes can be plugged with a resorbable material containing a large amount of a radiopaque agent, e.g. barium sulfate.

Other aspects, features, variations, and ways of using the present invention are described in the U.S. Patent Applications and filed under attorney docket numbers 030481/0249 (entitled “Closure Device”); 030481/0250 (entitled “Closure Device”); and 030481/0258 (entitled “Closure Device and Insertion Assembly”) concurrently herewith. The entire contents of these related applications are incorporated herein by reference. Features in these different applications may be combined with each other.

Although the present invention has been described with reference to specific embodiments, also shown in the appended drawings, it will be apparent for those skilled in the art that many variations and modifications can be done within the scope of the invention as described in the specification and defined with reference to the claims below. As mentioned above, it should in particular be noted that the lengths of the struts can be varied, such that, for example, the length of the proximal struts is longer than the length of the distal struts, or vice versa. As mentioned, it is possible to have different lengths of the articulated arms within a strut set, such that, for example, the distal arms are longer than the proximal arms, or vice versa. The weakened strut sections discussed above can be replaced with other designs that provide the desired hinge-like action. The hinge action could, for example, be accomplished by real hinges arranged along the struts.

What is claimed is:

1. A medical closure device having a longitudinal central axis and comprising:
a tubular member having a length and a first set of struts extending between a first end portion and a central portion and a second set of struts extending between said central portion and a second end portion, the struts being provided with a hinge section that acts as a hinge, such that said closure device is movable between a first elongated tubular introduction configuration and a second positioning configuration in which the first and second end portions have been moved relative to each other towards each other such that said hinge sections of the first and/or second sets of struts have moved radially away from said longitudinal central axis, and
the closure device further comprises a locking member, which has a first end rim with a diameter larger than a diameter of the first end portion and a second end rim with a diameter larger than a diameter of the second end portion, and the distance between the first and second end rims being smaller than the length of the tubular member, and
wherein the closure device has a third closed configuration in which the locking member is positioned in the tubular member such that the first end portion abuts the first end rim and an enlarged diameter of the second end rim prevents further compression of the tubular member, and
wherein the medical closure device has a fourth configuration in which the second end portion has been moved over the second end rim such that the closure device is held in an expanded and locked configuration.

2. The medical closure device according to claim 1, wherein the first set of struts is at least partly covered by a membrane.

3. The medical closure device according to claim 1, wherein the second set of struts is at least partly covered by a membrane.

4. The medical closure device according to claim 1, wherein said tubular member comprises two separate halves such that the respective halves can move independently of each other.

5. The medical closure device according to claim 1, wherein the closure device is at least partly made from a synthetic resorbable polymer.

6. The medical closure device according to claim 5, wherein said synthetic resorbable polymer is a polyester, a polyether ester, or a polycarbonate, or a mixture thereof.

7. The medical closure device according to claim 6, wherein the closure device is made from homopolymers or copolymers made from any of the monomers lactide, glycolide, epsilon-caprolactone, trimethylene carbonate, and paradioxanone.

8. The medical closure device according to claim 1, wherein said locking member constitutes a separate part of the closure device.

9. The medical closure device according to claim 8, wherein said locking member is made from a first material and said tubular member is made from a second material.

10. The medical closure device according to claim 9, wherein said first material is stiffer than said second material.

11. The medical closure device according to claim 9, wherein said first material has a longer resorption time than said second material.

12. The medical closure device according to claim 9, wherein said first material is made from homopolymers or copolymers where the main monomer component is lactide, caprolactone or paradioxanone.

13. The medical closure device according to claim 9, wherein said second material is made from a block copolymer characterized by having a soft middle part characterized by having a glass transition temperature below room temperature and a semi-crystalline part at each end of the soft middle part.

14. The medical closure device according to claim 13, wherein said semi-crystalline part is polymerized from any of the monomers glycolide, lactide and paradioxanone.

15. The medical closure device according to claim 9, wherein said second material is polyparadioxanone.

16. The medical closure device according to claim 1, wherein the closure device comprises a radiopaque agent.

17. The medical closure device according to claim 1, wherein the closure device is adapted for closing a septal defect in a heart.

18. The medical closure device according to claim 1, wherein the closure device is adapted for closing a puncture in a vessel wall.

19. A medical closure assembly comprising:
a medical closure device having a longitudinal central axis and comprising
a tubular member having a length and a first set of struts extending between a first end portion and a central portion and a second set of struts extending between said central portion and a second end portion, the struts being provided with a hinge section that acts as a hinge, such that said closure device is movable between a first elongated tubular introduction configuration and a second positioning configuration in which the first and second end portions have been moved relative to each other towards each other such that said hinge sections of the first and/or second sets of struts have moved radially away from said longitudinal central axis, and
the closure device further comprises a locking member, which has a first end rim with a diameter larger than a diameter of the first end portion and a second end rim with a diameter larger than a diameter of the second end portion, and the distance between the first and second end rims being smaller than the length of the tubular member, and
the closure device has a third closed configuration in which the locking member is positioned in the tubular member such that the first end portion abuts the first end rim and an enlarged diameter of the second end rim prevents further compression of the tubular member, and
wherein the medical closure assembly further comprises an actuator by which the second end portion can be moved over the second end rim such that the closure device is held in a fourth expanded and locked configuration.
